(19) United States
(12) Patent Application Publication
(10) Pub. No.: US 2010/0191029 A1
(43) Pub. Date: Jul. 29, 2010

(54) METHOD FOR PREPARATION OF LINEAR ALPHA-OLEFINS AND REACTOR SYSTEM THEREFOR

(76) Inventors: Peter M. Fritz, Unterhaching (DE); Heinz Bölt, Wolfratshausen (DE); Florian Winkler, Munich (DE); Wolfgang Müller, Munich (DE); Richard Schneider, Uffing (DE); Anton Wellenhofer, Munich (DE); Fuad Mousa, Riyadh (SA)

Correspondence Address: KRAMER LEVIN NAFTALIS & FRANKEL LLP;INTELLECTUAL PROPERTY DEPARTMENT, 1177 AVENUE OF THE AMERICAS, NEW YORK, NY, 10036, US

(21) Appl. No.: 12/448,934

(22) PCT Filed: Nov. 19, 2007
(86) PCT No.: PCT/EP2007/009979

§ 371 Date: Apr. 13, 2010

(30) Foreign Application Priority Data
Jan. 19, 2007 (EP) 07001109.3

(51) Int. Cl. C07C 2/30; C07C 2/08; C07C 2/26; B01J 19/00

(52) U.S. Cl. 585/522; 585/520; 585/523; 422/198

(57) ABSTRACT

The present invention relates to a method for the preparation of linear alpha-olefins by oligomerization of ethylene in a reactor in the presence of a catalyst and solvent, wherein an outlet stream from the reactor comprising the solvent, catalyst and linear alpha-olefins is heated by at least one heating means to a temperature at which all or substantially all of the linear alpha-olefins are dissolved and/or melted in the outlet stream; and a reactor system therefor.

The present invention relates to a method for the preparation of linear alpha-olefins by oligomerization of ethylene in a reactor in the presence of a catalyst and solvent and a reactor system therefore.

Methods for the preparation of linear alpha-olefins are widely known in the art. The methods are carried out in a reactor, usually in the presence of a catalyst, wherein an outlet stream is discharged from the reactor comprising solvent, catalyst and linear alpha-olefins. As is common knowledge, the linear alpha-olefins may comprise olefins having short chains, i.e. having 4 to 18 carbon atoms, which are liquid under oligomerization conditions, but may also comprise olefins having 20 or more carbon atoms, which are usually solid or semi-solid under oligomerization conditions, especially after discharge from the reactor for transferring the outlet stream to a catalyst removal section for deactivating and removing the catalyst.

Especially under start-up conditions and at extremely low ambient temperatures, thus, a deposition of heavy molecular weight oligomers, especially in the catalyst removal section, can take place. The consequence is plugging of equipment and piping systems.

Additionally, it is known that for some prior art methods cooling and solidification of high molecular weight oligomers is intended at the outlet of the oligomerization reactor for removal thereof. However, this is a complicated and operational-extensive mode of operation.

It is an object of the present invention to provide a method for the preparation of linear alpha-olefins which overcomes the drawbacks of the prior art. Especially a method shall be provided wherein deposition of heavy molecular weight oligomers can be avoided, as desired, especially in a catalyst removal section and in equipment and piping systems downstreams thereof.

Further a reactor system for carrying out such a method shall be provided.

The first object is achieved in that an outlet stream from the reactor comprising the solvent, catalyst and linear alpha-olefins is heated by at least one heating means to a temperature to allow substantially all of the linear alpha-olefins to be dissolved and/or melted in the outlet stream.

Preferably the temperature is from about 80 to about 140° C.

More preferred, the linear alpha-olefins comprise olefins having 4 to more than 20 carbon atoms.

Heating may be achieved by electrical tracing, steam tracing or a heat exchanger.

Preferably, heating is carried out in a catalyst removal section where catalyst is deactivated and removed.

In one embodiment, heating is achieved by addition of heated caustic deactivation agent within the catalyst removal section.

The temperature downstreams of the catalyst removal section can be preferably kept to be not lower than in the catalyst removal section until final disposal of undesired components of the outlet stream.

Here it is preferred that non-desired components are catalyst constituents and/or linear alpha-olefins having more than 20 carbon atoms.

More preferred, the final disposal is from a bottoms of a separation column.

Even preferred, an oligomerization is carried out utilizing a homogeneous catalyst comprising a zirconium component and an organoaluminum component.

Moreover, it is preferred that the zirconium component has the formula ZrCl₄₋ₘXₘ, wherein X═OCOR or OSO₃R′ with R and R′ being independently alkyl, alkene and phenyl, and wherein 0<m<4.

In one embodiment, the organoaluminum component is Al(C₂H₅)₃, Al₂Cl₃(C₂H₅)₃, AlCl(C₂H₅)₂ or a mixture thereof.

Preferably, the residence time of the alpha olefin stream from the reactor outlet to the catalyst removal section is below 2 minutes.

According to the invention is also a reactor system for the preparation of linear alpha-olefins, especially by a method according to the present invention, comprising a reactor having an outlet for discharging an outlet stream comprising solvent, catalyst and linear alpha-olefins, and heating means to heat the outlet stream from the reactor.

Preferably, the heating means is placed in a catalyst removal section connected to the reactor.

Finally, the catalyst removal section is preferably adjacent to the reactor outlet, in order to maintain the residence time of the alpha olefin stream from the reactor outlet to the catalyst removal section below 2 minutes.

Surprisingly, it was found that by provision of heating means in a reactor system for preparing linear alpha-olefins, preferably directly adjacent to the reactor, deposition of heavy molecular weight oligomers can be avoided, and therefore plugging of equipment and piping systems. In the inventive method it is important that the temperature of the outlet stream is adjusted to allow substantially all of the linear alpha-olefins, including heavy molecular weight oligomers, to be dissolved and/or melted therein.

This temperature should be maintained until final disposal of non-desired components of this outlet stream, in order to avoid plugging of equipment and piping systems downstreams the heating means. Preferably, heating is carried out in the catalyst removal section which is preferably immediately adjacent to the reactor outlet in order to deactivate the catalyst as soon as possible to avoid further oligomerization in reactor piping and equipment.

It is obvious that the inventive method is not necessarily restricted to the oligomerization of ethylene to obtain linear alpha-olefins, but may be utilized in all technologies handling high molecular weight oligomers.

Additional features and advantages of the present invention are now illustrated in detail with reference to the accompanying drawing wherein

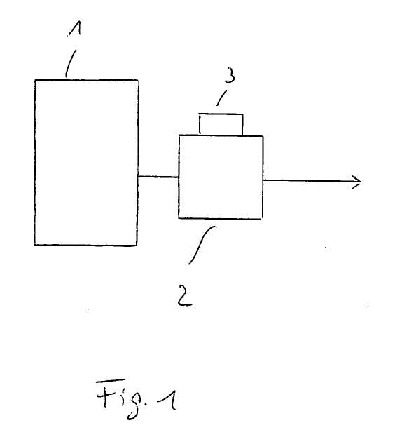

FIG. 1 shows a schematic illustration of the inventive method for the preparation of linear alpha-olefins.

In FIG. 1 a reactor 1 is shown which can be utilized for the oligomerization of ethylene to prepare linear-alpha olefins. In the reactor 1 ethylene is oligomerized in the presence of solvent and catalyst, preferably at a temperature of about 60-100° C. After oligomerization (the reactor is preferably operated continuously), an outlet stream is removed from the reactor. The outlet stream comprises the solvent, catalyst and linear alpha-olefins, either with low and high molecular weight. In detail, the term “high molecular weight oligomer” is meant to comprise oligomers having such a high molecular weight that they are substantially solid at reaction temperature. The outlet stream from the reactor 1 is then preferably transferred to a catalyst removal section 2 where catalyst is deactivated and removed by addition of a catalyst deactivation agent, e.g. a solution of sodium hydroxide. It is preferred that the residence time of the outlet stream from the reactor to the catalyst removal section is as short as possible, preferably below 2 minutes. At the catalyst removal section 2, there is also provided a heating means 3 to heat the outlet stream to a temperature so that the linear alpha-olefins are substantially all dissolved and/or melted in the outlet stream. Someone skilled in the art is aware of any heating means which can be utilized for this purpose. After the catalyst removal section 2 the outlet stream can be further processed, e.g. the linear alpha-olefins may be separated from solvent and catalyst residues. Especially, the linear alpha-olefins may be separated into several fractions; e.g. into fractions having low and high molecular weight. The fraction of high molecular weight oligomers may be finally disposed, preferably from a bottoms of a separation column. It is evident for someone skilled in the art that there may be provided additional heating means downstreams of the catalyst removal section to keep the temperature of the outlet stream to be not lower than in the catalyst removal section, so that deposition of high molecular weight oligomers may be avoided in additional equipment and pipings.

The features disclosed in the foregoing description, in the drawing or in the claims may, both separately and in any combination thereof, be material for realizing the invention in diverse forms thereof.

1. A method for the oligomerization of ethylene in a reactor in the presence of a catalyst and solvent at oligomerization conditions to produce a reaction product comprising linear alpha-olefins, the solvent, and the catalyst, wherein at least a portion of the linear alpha-olefins in the reaction product are solid or semi-solid at the oligomerization conditions, comprising heating said reaction product to a first temperature at which all or substantially all of the linear alpha-olefins are dissolved and/or melted in the reaction product.

2. (canceled)

3. (canceled)

4. The method according to claim 1, wherein the heating of the reaction production is done using electrical tracing, steam tracing or a heat exchanger.

5. The method according to claim 1, comprising the further step of deactivating the catalyst in the reaction product.

6. The method according to claim 5, wherein the reaction product is heated by the addition of a catalyst deactivation agent during the catalyst deactivation step.

7. The method according to claim 5, wherein the reactor product comprises low molecular weight linear-alpha olefins and high molecular weight linear alpha-olefins and the reactor product is separated into at least a low molecular weight fraction and high molecular weight fraction after the catalyst deactivation step.

8. The method according to claim 7, wherein the reactor product is maintained at a second temperature at which all or substantially all of the linear alpha-olefins therein are dissolved and/or melted until at least the low and high molecular weight linear alpha-olefins are separated.

9. The method according to claim 5, wherein the residence time of the reaction product between the reactor and the catalyst deactivation step is less than 2 minutes.

10. The method according to claim 1, wherein the catalyst comprises a zirconium component and an organoaluminum component.

11. The method according to claim 10, wherein the zirconium component has the formula ZrCl₄₋ₘXₘ, wherein X═OCOR or OSO₃R′ with R and R′ being independently of alkyl, alkene and phenyl, and wherein 0<m<4.

12. The method according to claim 11, wherein the organoaluminum component comprises Al(C₂H₅)₃, Al₂Cl₃(C₂H₅)₃, AlCl(C₂H₅)₂ or a mixture thereof.

13. The method according to claim 12, comprising the further step of deactivating the catalyst in the reaction product and wherein the residence time of the the reactor product between the reactor and catalyst deactivation step is less than 2 minutes.

14. A reactor system for the oligomerization of ethylene in the presence of a catalyst and a solvent, comprising a reactor which produces a reaction product comprised of low molecular weight linear alpha-olefins, high molecular weight linear alpha-olefins, the catalyst and the solvent and a reaction product heater adapted to increase the temperature of the reaction product to a temperature at which all or substantially all of the linear alpha-olefins in the reaction product are dissolved or melted.

15. The reactor system according to claim 14, further comprising a catalyst deactivation unit for receiving the reaction product and deactivating the catalyst therein, wherein the reaction product heaters is integral to the catalyst deactivation unit.

16. The reactor system according to claim 15, wherein the catalyst removal unit receives the reactor product directly from the reactor.

17. The reactor system according to claim 16, wherein the residence time of the the reactor product between the reactor and to the catalyst removal unit is less than 2 minutes.

18. The method according to claim 12, wherein the reaction product comprises linear alpha-olefins having 4 carbon atoms to more than 20 carbon atoms, and wherein the first temperature is in the range of about 80° C. to about 140° C.
